(19) United States
(12) Patent Application Publication
(10) Pub. No.: US 2011/0245571 A1
(43) Pub. Date: Oct. 6, 2011

(54) Oxidative dehydrogenation of paraffins field of the invention

(75) Inventors: Leonid Modestovich Kustov, Moscow (RU); Aleksey Victorovich Kucherov, Moscow (RU); Elena Dmitrievna Finashina, Moscow (RU); Alexander Yurievich Stakheev, Moscow (RU); Ilya Mikhailovich Sinev, Moscow (RU); Andrzej Krzywicki, Calgary (CA)

(73) Assignee: NOVA Chemicals (International) S.A.

(21) Appl. No.: 13/065,241

(22) Filed: Mar. 17, 2011

(30) Foreign Application Priority Data
Mar. 31, 2010 (CA) 2,699,836

(51) Int. Cl. C07C 5/333

(52) U.S. Cl. 585/658

(57) ABSTRACT

The present invention provides a process for the oxidative dehydrogenation of a paraffin such as ethane to the corresponding alkene such as ethylene in which the alkane is contacted with a bed of oxidative dehydrogenation catalyst having an enhanced labile oxygen content in the crystal structure on an inert support optionally with a regenerable metallic oxidant composition in the absence of a gaseous feed containing oxygen.

### FIELD OF THE INVENTION

The present invention relates to the oxidative dehydrogenation of paraffins to olefins. More particularly, the present invention relates to the catalytic oxidative dehydrogenation of paraffins to olefins in the presence of a catalyst having an enhanced oxygen storage capacity. The catalyst of the present invention may be used with air as an oxidant.

### BACKGROUND OF THE INVENTION

Currently paraffins, particularly aliphatic paraffins, are converted to olefins using thermal cracking technology. Typically the paraffins are passed through a furnace tube heated to at least 800° C., typically from about 850° C. to the upper working temperature of the alloy for the furnace tube, generally about 950° C. to 1000° C., for a period of time in the order of milliseconds to a few seconds. The paraffin molecule loses hydrogen and one or more unsaturated bonds are formed to produce olefins and/or dienes. The current thermal cracking processes are not only cost intensive to build and operate but also energy intensive due to the substantial heat requirement for the endothermic cracking reactions. As a result, significant amounts of CO₂ are produced from the operation of these cracking furnaces.

Alternatively, it is known that olefins can be produced by reactions between paraffins with oxygen. However, this technology has not been commercially practiced for a number of reasons including the potential for an explosive mixture of oxygen and paraffin at an elevated temperature. For satisfactory conversion of paraffins to olefins, the required oxygen in the feed mixture should be typically higher than the maximum allowable level before entering the explosion range. Another reason is the requirement of either front, end oxygen separation (from air) or a back end nitrogen separation, which often brings the overall process economy into negative territory. Therefore, solutions to address these issues are being sorted in various directions. However, if there were an oxidative dehydrogention catalyst available with a greater oxygen capacity it would reduce or eliminate the need for front end oxygenation separation thus reducing the capital and operating costs of the process.

There are a number of U.S. patents assigned to Petro-Tex Chemical Corporation issued in the late 1960's that disclose the use of various ferrites in a steam cracker to produce olefins from paraffins. The patents include U.S. Pat. Nos. 3,420,911 and 3,420,912 in the names of Woskow et al. The patents teach introducing ferrites such as zinc, cadmium, and manganese ferrites (i.e. mixed oxides with iron oxide). The ferrites are introduced into a dehydrogenation zone at a temperature from about 250° C. up to about 750° C. at pressures less than 100 psi (689.476 kPa) for a time less than 2 seconds, typically from 0.005 to 0.9 seconds. Preferably the feed is a mixture of not less than 50 mole % mono olefins and optionally paraffins (e.g. the preferred product is a diene). The reaction appears to take place in the presence of steam that may tend to shift the equilibrium in the “wrong” direction. Additionally, the reaction takes place in the presence of a catalyst not of the type of the present invention.

In the Petro-Tex patents the metal ferrite (e.g. MFeO₄ where, for example, M is Mg, Mn, Co, Ni, Zn or Cd) is circulated through the dehydrogenation zone and then to a regeneration zone where the ferrite is reoxidized and then fed back to the dehydrogenation zone.

The patent GB 1,213,181, which seems to correspond in part to the above Petro-Tex patents, discloses that nickel ferrite may be used in the oxidative dehydrogenation process. The reaction conditions are comparable to those of above noted Petro-Tex patents.

Subsequent to the Petro-Tex patents a number of patents were published relating to the catalytic dehydrogenation of paraffins. However, these patents do not include the use of the ferrites of the Petro-Tex patents to provide a source of oxygen.

Several catalytic systems are known in the art for the oxidative dehydrogenation of ethane. U.S. Pat. No. 4,450,313, issued May 22, 1984 to Eastman et al., assigned to Phillips Petroleum Company discloses a catalyst of the composition LiO—TiO₂, which is characterized by a low ethane conversion not exceeding 10%, in spite of a rather high selectivity to ethylene (92%). The major drawback of this catalyst is the high temperature of the process of oxidative dehydrogenation, which is close to or higher than 650° C.

The U.S. Pat. Nos. 6,624,116, issued Sep. 23, 2003 to Bharadwaj et al. and 6,566,573 issued May 20, 2003 to Bharadwaj et al., both assigned to Dow Global Technologies Inc., disclose Pt—Sn—Sb—Cu—Ag monolith systems that have been tested in an autothermal regime at T>750° C., the starting gas mixture contained hydrogen (H₂:O₂=2:1, GHSV=180 000 h⁻¹). The catalyst composition is different from that of the present invention and the present invention does not contemplate the use of molecular hydrogen in the feed.

U.S. Pat. Nos. 4,524,236 issued Jun. 18, 1985 to McCain, assigned to Union Carbide Corporation and 4,899,003, issued Feb. 6, 1990 to Manyik et al., assigned to Union Carbide Chemicals and Plastics Company Inc., disclose mixed metal oxide catalysts of V—Mo—Nb—Sb. At 375-400° C. the ethane conversion reached 70% with the selectivity close to 71-73%. However, these parameters were achieved only at very low gas hourly space velocities less than 900 h⁻¹ (i.e. 7201⁻¹).

Rather promising results were obtained for nickel-containing catalysts disclosed in U.S. Pat. No. 6,891,075, issued May 10, 2005 to Liu, assigned to Symyx technologies, Inc. At 325° C. the ethane conversion on the best catalyst in this series was about 20% with a selectivity of 85% (a Ni—Nb—Ta oxide catalyst). The patent teaches a catalyst for the oxidative dehydrogenation of a paraffin (alkane) such as ethane. The gaseous feedstock comprises at least the alkane and oxygen, but may also include diluents (such as argon, nitrogen, etc.) or other components (such as water or carbon dioxide). The dehydrogenation catalyst comprises at least about 2 weight % of MO and a broad range of other elements preferably Nb, Ta, and Co. While NiO is present in the catalyst it does not appear to be the source of the oxygen for the oxidative dehydrogenation of the alkane (ethane).

U.S. Pat. No. 6,521,808 issued Feb. 18, 2003 to Ozkan, et al., assigned to the Ohio State University teaches sol gel supported catalysts for the oxidative dehydrogenation of ethane to ethylene. The catalyst appears to be a mixed metal system such as Ni—Co—Mo, V—Nb—Mo possibly doped with small amounts of Li, Na, K, Rb, and Cs on a mixed silica oxide/titanium oxide support. Again the catalyst does not provide the oxygen for the oxidative dehydrogenation; rather gaseous oxygen is included in the feed.

U.S. Pat. No. 7,319,179 issued Jan. 15, 2008 to Lopez-Nieto et al., assigned to Consejo Superior de Investigaciones Cientificas and Universidad Politecnica de Valencia, discloses Mo—V—Te—Nb—O oxide catalysts that provided an ethane conversion of 50-70% and selectivity to ethylene up to 95% (at 38% conversion) at 360-400° C. The catalysts have the empirical formula MoTeₕVᵢNbⱼAₖOₓ, where A is a fifth modifying element. The catalyst is a calcined mixed oxide (at least of Mo, Te, V and Nb), optionally supported on: (i) silica, alumina and/or titania, preferably silica at 20-70 wt % of the total supported catalyst or (ii) silicon carbide. In the examples the high yield catalyst has carbon as a fifth element. This is not present in the catalyst of the present invention. The catalysts of the present invention have a higher selectivity and a higher time space yield than those of Lopes Nieto.

Similar catalysts have been also described in open publications of Lopez-Nieto and co-authors. Selective oxidation of short-chain alkanes over hydrothermally prepared MoVTeNbO catalysts is discussed by F. Ivars, P. Botella, A. Dejoz, J. M. Lopez-Nieto, P. Concepcion, and M. I. Vazquez, in Topics in Catalysis (2006), 38(1-3), 59-67.

MoVTe—Nb oxide catalysts have been prepared by a hydrothermal method and tested in the selective oxidation of propane to acrylic acid and in the oxidative dehydrogenation of ethane to ethylene. The influence of the concentration of oxalate anions in the hydrothermal gel has been studied for two series of catalysts, Nb-free and Nb-containing, respectively. Results show that the development of an active and selective active orthorhombic phase (Te₂M₂₀O₅₇, M=Mo, V, Nb) requires an oxalate/Mo molar ratio of 0.4-0.6 in the synthesis gel in both types of samples. The presence of Nb favors a higher catalytic activity in both ethane and propane oxidation and a better production of acrylic acid.

Mixed metal oxide supported catalyst compositions, catalyst manufacture and use in ethane oxidation are described in Patent WO 2005018804 A1, 3 Mar. 2005, assigned to BP Chemicals Limited, UK. A catalyst composition for the oxidation of ethane to ethylene and acetic acid comprises (i) a support and (ii) in combination with O, the elements Mo, V and Nb, optionally W and a component Z, which is ≦1 metals of Group 14. Thus, Mo_{60.5}V₃₂Nb_{7.5}Oₓ on silica was modified with 0.33 g-atom ratio Sn for ethane oxidation with good ethylene/acetic acid selectivity and product ratio 1:1.

A process for preparation of ethylene from gaseous feed comprising ethane and oxygen involving contacting the feed with a mixed oxide catalyst containing vanadium, molybdenum, tantalum and tellurium in a reactor to form an effluent of ethylene is disclosed in WO 2006130288 A1, 7 Dec. 2006, assigned to Celanese Int. Corp. The catalyst has selectivity for ethylene of 50-80% thereby allowing oxidation of ethane to produce ethylene and acetic acid with high selectivity. The catalyst has the formula Mo₁V_{0.3}Ta_{0.1}Te_{0.3}O_{z}. The catalyst is optionally supported on a support selected from porous silicon dioxide, fused silica, kieselguhr, silica gel, porous and nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, aluminum silicate, silicon nitride, silicon carbide, and glass, carbon, carbon-fiber, activated carbon, metal-oxide or metal networks and corresponding monoliths; or is encapsulated in a material (preferably silicon dioxide (SiO₂), phosphorus pentoxide (P₂O₅), magnesium oxide (MgO), chromium trioxide (Cr₂O₃), titanium oxide (TiO₂), zirconium oxide (ZrO₂) or alumina (Al₂O₃).

The preparation of a supported catalyst usable for low temperature oxy-dehydrogenation of ethane to ethylene is disclosed in the U.S. Pat. No. 4,596,787 A, 24 Jun. 1986 assigned to UNION CARBIDE CORP. A supported catalyst for the low temperature gas phase oxydehydrogenation of ethane to ethylene is prepared by (a) preparing a precursor solution having soluble and insoluble portions of metal compounds; (b) separating the soluble portion; (c) impregnating a catalyst support with the soluble portion and (d) activating the impregnated support to obtain the catalyst. The calcined catalyst has the composition MoₐV_{b}Nb_{c}Sb_{d}Xₑ. X is nothing or Li, Sc, Na, Be, Mg, Ca, Sr, Ba, Ti, Zr, Hf, Y, Ta, Cr, Fe, Co, Ni, Ce, La, Zn, Cd, Hg, Al, Tl, Pb, As, Bi, Te, U, Mn and/or W; a is 0.5-0.9, b is 0.1-0.4, c is 0.001-0.2, d is 0.001-0.1, e is 0.001-0.1 when X is an element. The catalyst does not appear to have the high conversion of the catalyst of the present invention.

Another example of the low temperature oxy-dehydrogenation of ethane to ethylene using a calcined oxide catalyst containing molybdenum, vanadium, niobium and antimony is described in the U.S. Pat. Nos. 4,524,236 A, 18 Jun. 1985 and 4,250,346 A, 10 Feb. 1981, both assigned to UNION CARBIDE CORP. The calcined catalyst contains MoₐV_{b}Nb_{c}Sb_{d}Xₑ in the form of oxides. The catalyst is prepared from a solution of soluble compounds and/or complexes and/or compounds of each of the metals. The dried catalyst is calcined by heating at 220-550° C. in air or oxygen. The catalyst precursor solutions may be supported on to a support, e.g. silica, aluminum oxide, silicon carbide, zirconia, titania or mixtures of these. The selectivity to ethylene may be greater than 65% for a 50% conversion of ethane.

The present invention seeks to provide a simple process for the oxidative dehydrogenation of paraffins in the absence of a gaseous feed of oxygen or an oxygen containing gas, in the presence of a catalyst having an enhanced ability to store oxygen and optionally a metal oxide or a mixture of metal oxides to provide oxygen for the catalytic process. The catalyst and/or oxide may be regenerated and used again either by recycling through a regeneration zone or by using parallel beds so that the catalyst and/or oxide may be regenerated by swinging the feed from an exhausted bed to a fresh bed and regenerating the catalyst and/or oxide in the exhausted bed. However, due to the enhanced oxygen capacity of the oxidative dehydrogenation catalyst of the present invention it is not necessary to use the catalyst in conjunction with the oxide.

### SUMMARY OF THE INVENTION

The present invention provides a process for the oxidative dehydrogenation of one or more C₂₋₁₀ alkanes to the corresponding C₂₋₁₀ alkene at a selectivity of greater than 95%, comprising contacting said alkane in the absence of a gaseous oxygen with a moving or fluid particulate bed of oxidative dehydrogenation catalyst having an enhanced labile oxygen content in the crystal structure on an inert support optionally with a regenerable metal oxide composition at a temperature from 300° C. to 700° C., a pressure from 0.5 to 100 psi (3.447 to 689.47 kPa) and a residence time of the alkane in said bed from 1 to 60 seconds, wherein the oxidative dehydrogenation catalyst is selected from the group consisting of

i) catalysts of the formula:

VₓMoₐNb_{b}Te_{c}Me_{d}Oₑ,

wherein
Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.1 to 0.9;
a is from 0.001 to 0.5;
b is from 0.001 to 0.5;
c is from 0.001 to 0.5;
d is from 0.001 to 0.5; and
e is a number to satisfy the valence state of the mixed oxide catalyst and regenerating at least one of the labile oxygen content in the crystal structure of the oxidative dehydrogenation catalyst and the metal oxide if present.

In a further embodiment the temperature of the oxidative dehydrogenation process is from 350° C. to 500° C., the pressures is from 15 to 50 psi (103.4 to 344.73 kPa) and the residence time of the alkane in said bed is from 5 to 20 seconds.

In a further embodiment the oxidative dehydrogenation catalyst is on a support selected from the group consisting of oxides of titanium, zirconium, aluminum, magnesium, yttrium, lanthanum, silicon and their mixed compositions or a carbon matrix at a loading from 1 to 95 weight % of the supported oxidative dehydrogenation catalyst.

In a further embodiment the oxidative dehydrogenation catalyst has selectivity for the corresponding 1-alkene of greater than 95%.

In a further embodiment the space-time yield of alkene in g/hour per Kg of catalyst is greater than 900 g/hour per kg of oxidative dehydrogenation catalyst.

In a further embodiment the alkane is selected from the group consisting of C₂₋₄ straight chained alkanes.

In a further embodiment the regeneration of the catalyst and metal oxide when present takes place at temperatures from 200° C. to 600° C., at pressures less than 10132.5 kPa (100 atm, 14700 psi) and the gaseous feed stream for the regeneration is selected from the group consisting of air, oxygen or a mixture of about 10 to 45 wt % oxygen and from 90 to 55 wt % of an inert gas.

In a further embodiment the alkane is ethane and in the catalyst x is from 0.02 to 0.5, a is from 0.1 to 0.45, b is from 0.1 to 0.45, c is from 0.1 to 0.45, and d is from 0.1 to 0.45.

In a further embodiment there are two or more separate fixed beds in parallel arrangement and one or more beds is regenerated by passing air there through while maintaining at least one bed in operation.

In a further embodiment the bed is a fluidized bed or a simple moving bed and a portion of the bed is removed from the reactor and regenerated by passing air there through and the regenerated bed is returned to the reactor.

In a further embodiment the metal oxide is present and is selected from the group consisting of NiO, Ce₂O, Ce₂O₃, Fe₂O₃, TiO₂, Cr₂O₃, V₂O₅, WO₃, and ferrites of the formula MFeO₄ where M is selected from the group consisting of Mf, Mn, Co, Ni, Zn, or Cd, and alumina and mixtures and is present in an amount to provide a weight ratio of oxidative dehydrogenation catalyst to metal oxide from 0.8:1 to 1:0.8.

In a further embodiment the bed is a segregated bed with the metal oxide separated from the oxidative dehydrogenation catalyst by a screen or an oxygen permeable membrane and at least a portion of the metal oxide is removed from said bed and regenerated by passing an oxygen containing gas stream there through and the metal oxide is returned to the bed.

The present invention also contemplates combinations of the foregoing embodiments in whole or in part and singularly and in combinations including an aggregate combination of all the embodiments.

### BRIEF DESCRIPTION OF THE DRAWINGS

FIG. 1 is a schematic drawing of a moving bed oxidative dehydrogenation process of the present invention.

### DETAILED DESCRIPTION

In the present specification the terms catalyst, support and metal oxide have been used in a fairly conventional manner. However, upon reading the specification it will be apparent to one of ordinary skill in the art that the components may serve several functions. For example alumina may be a support and a metal oxide. Further some of the metal oxides, such as ferrites, may act as catalyst for the oxidative dehydrogenation (albeit at a rate less than the preferred catalysts). The inventors intend that the specification be given a broad purposeful construction recognizing that some of the components used in accordance with the present invention may serve multiple concurrent capacities.

In the supported catalyst of the present invention the active phase (the catalyst) is used in an amount from 1 to 95, preferably 10 to 95, most preferably from 30 to 80, desirably from 40 to 70 weight % of the supported catalyst and the support is present in an amount from 99 to 5 preferably from 90 to 5, most preferably from 70 to 20, desirably from 60 to 30 weight % of the total catalyst.

The catalyst has the formula:

VₓMoₐNb_{b}Te_{c}Me_{d}Oₑ,

wherein Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd, Pt, Ag, Cd, Os, Ir, Au, and mixtures thereof; and
x is from 0.1 to 0.9, preferably from 0.2 to 0.5;
a is from 0.001 to 0.5, preferably from 0.1 to 0.45;
b is from 0.001 to 0.5, preferably from 0.1 to 0.45;
c is from 0.001 to 0.5, preferably from 0.1 to 0.45;
d is from 0.001 to 0.5, preferably from 0.1 to 0.45; and
e is a number to satisfy the valence state of the mixed oxide catalyst.

In the above formula the numbers represent the molar amounts of the components. Preferably the ratio of x:c is from 0.3 to 10, most preferably from 0.5 to 8, desirably from 0.5 to 6.

The active metal catalyst may be prepared by mixing aqueous solutions of soluble metal compounds such as hydroxides, sulphates, nitrates, halides, lower (C₁₋₅) mono- or dicarboxylic acids, and ammonium salts or the metal-containing acid per se. For instance, the catalyst could be prepared by blending solutions such as ammonium metavanadate, niobium oxalate, ammonium molybdate, telluric acid etc. The resulting solution is then dried typically in air at 100-150° C. and calcined in a flow of inert gas such as those selected from the group consisting of N₂, He, Ar, Ne and mixtures thereof at 200-600° C., preferably at 300-500° C. The calcining step may take from 1 to 20, typically from 5 to 15 usually about 10 hours. The resulting oxide is a friable solid.

The support for the catalyst may be selected from the group consisting of porous silicon dioxide, fused silicon dioxide, kieselguhr, silica gel, porous and nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, yttrium oxide, aluminum silicate, silicon nitride, silicon carbide, and glass, carbon, carbon-fiber, activated carbon, metal-oxide or metal networks and corresponding monoliths; or is encapsulated in a material (preferably silicon dioxide (SiO₂), magnesium oxide (MgO), chromium trioxide (Cr₂O₃), titanium oxide (TiO₂), zirconium oxide (ZrO₂) or alumina (Al₂O₃).

Preferred supports include oxides of titanium, zirconium, aluminum, magnesium, yttrium, lanthanum, silicon and their mixed compositions or a carbon matrix. The support may have a broad range of surface area, typically greater than 25 m²/g up to 1,000 m²/g. High surface area supports may have a surface area greater than 250 m²/g (e.g. from 250 to 1,000 m²/g). Low to moderate surface area supports may have a surface area from 25 to 250 m²/g, preferably from about 50 to 200 m²/g. It is believed the higher surface area supports will produce more CO₂ during the oxidative dehydrogenation of the alkane.

The support will be porous and may have a pore volume up to about 5.0 ml/g, preferably less than 3 ml/g typically from about 0.1 to 1.5 ml/g, preferably from 0.15 to 1.0 ml/g.

It is also believed that titanium silicates such as those disclosed in U.S. Pat. No. 4,853,202 issued Aug. 1, 1989 to Kuznicki, assigned to Engelhard Corporation, would be useful as supports in accordance with the present invention.

It is important that the support be dried prior to use. Generally, the support may be heated at a temperature of at least 200° C. for up to 24 hours, typically at a temperature from 500° C. to 800° C. for about 2 to 20 hours, preferably 4 to 10 hours. The resulting support will be free of adsorbed water and should have a surface hydroxyl content from about 0.1 to 5 mmol/g of support, preferably from 0.5 to 3 mmol/g per gram of support.

The amount of the hydroxyl groups in silica may be determined according to the method disclosed by J. B. Peri and A. L. Hensley, Jr., in J. Phys. Chem., 72 (8), 2926, 1968, the entire contents of which are incorporated herein by reference.

The support and catalyst may be combined and then comminuted to produce a fine particulate material having a particle size ranging from 1 to 100 micron. The comminution process may be any conventional process including ball and bead mills, rotary, stirred and vibratory, bar or tube mills, hammer mills, and grinding discs. A preferred method of comminution is a ball or bead mill.

In one embodiment of the invention the catalyst and the support are dry milled. It is also possible to wet mill the catalyst and support provided the resulting product is again dried and if necessary calcined.

The particulate material may be sieved if required to select the appropriate small particle size. The particulates may then be compacted and crushed to yield particles having a size from 0.1 to 1-2 mm. The particles or extrudates can be formed that can be further loaded in the catalytic reactor

The oxidative dehydrogenation may be conducted at temperatures from 300° C. to 700° C., typically from 300° C. to 600° C., preferably from 350° C. to 500° C., at pressures from 0.5 to 100 psi (3.447 to 689.47 kPa), preferably from 15 to 50 psi (103.4 to 344.73 kPa) and the residence time in the reactor is typically from 2 to 30 seconds preferably from 5 to 20 seconds. The paraffin (alkane) may be a C₂₋₈, preferably a C₂₋₄ straight chained paraffin. The paraffin feed should be of purity of preferably 95%, most preferably 98% of the same paraffin. Preferably the paraffin is a high purity ethane. Preferably the process has selectivity for the alkene or diene, preferably 1-alkene from the corresponding alkane of greater than 95%, preferably greater than 98%. The gas hourly space velocity (GHSV) will be from 900 to 18000 h⁻¹, preferably greater than 1000 h⁻¹. The space-time yield of alkene (e.g. ethylene) (productivity) in g/hour per Kg of catalyst should be not less than 900, preferably greater than 1500, most preferably greater than 3000, most desirably greater than 3500 at 350° C. It should be noted that the productivity of the catalyst will increase with increasing temperature.

The reactor may be a plug flow reactor or a fluidized bed reactor. In these embodiments of the invention a portion of exhausted catalyst and optionally metal oxide, when present, is removed from the bed and regenerated and then returned to the bed.

The regeneration of the catalyst and metal oxide when present typically takes place at temperatures from 200° C. to 600° C., preferably from about 300° C. to about 550° C., desirably from 400° C. to 450° C., at pressures less than 10132.5 kPa (100 atm, 1470.0 psi), typically less than 5066.25 kPa (50 atm 735.0 psi), desirably from 1013.25 kPa (10 atm 147 psi) to 101.32 kPa (1 atm 14.7 psi). The gaseous feed stream for the regeneration may be air, oxygen or a mixture of about 10 to 45 wt % oxygen and from 90 to 55 wt % of an inert gas such as nitrogen, helium, argon, or a mixture thereof. From an industrial point of view air is preferable for the feed stream to regenerate the catalyst and the metal oxide when present. The time to regenerate the catalyst and metal oxide when present will depend on the mass of the material to be regenerated and the space velocity of the regenerant (air, oxygen etc.). This may be easily determined by one of ordinary skill in the art using routine non inventive testing of small samples of the material to be regenerated.

In one embodiment the bed (oxidative dehydrogenation catalyst optionally with a metal oxide) is a fluidized bed or a simple moving bed, and a portion of the bed is removed from the reactor and regenerated by passing air there through and the regenerated bed is returned to the reactor.

In a further embodiment of the invention the reactor may comprise several beds in parallel so that one or more beds may be used in the reaction while one or more beds may be regenerated in situ without the alkane present, under conditions as described above.

In a further embodiment of the invention the supported catalyst may be used in conjunction with a metal oxide that provides the source of oxygen for the oxidative dehydrogenation, which may be NiO, CeO₂, Ce₂O₃, Fe₂O₃, TiO₂, Cr₂O₃, V₂O₅, WO₃, rare earth oxides, ferrites of the formula MFeO₄ where, for example, M is selected from the group consisting of Mg, Mn, Co, Ni, Zn or Cd, and mixtures thereof and the weight ratio of oxidative dehydrogenation catalyst to metal oxide is from 0.8:1 to 1:0.8. In a further embodiment of the invention the metal oxide is a mixture of NiO, Ce₂O, Ce₂O₃, Fe₂O₃, TiO₂, Cr₂O₃, V₂O₅, WO₃, rare earth oxides, ferrites of the formula MFeO₄ where, for example, M is selected from the group consisting of Mg, Mn, Co, Ni, Zn or Cd, and alumina in a weight ratio 0.8:1 to 1:0.8 and the oxidative dehydrogenation catalyst is used in an amount to provide a weight ratio of oxidative dehydrogenation catalyst to metal oxide from 0.8:1 to 1:0.8.

In the embodiments where a metal oxide is present, the regeneration of the metal oxide is performed as described above.

However, other embodiments are also possible. For example the reactor could comprise a chamber separated by one or several fine screens. The supported oxidative dehydrogenation catalyst would be on one side of the fine screen and the metal oxide on the other side of the fine screen so oxygen could be transported from the metal oxide across the screen to the oxidative dehydrogenation catalyst. In this type of embodiment only the metal oxide need to be regenerated by direct contact with the oxygen containing gas (i.e. there is no direct feed of an oxygen contain gas to the bed containing the oxidative dehydrogenation catalyst). Although one could regenerate both the oxidative dehydrogenation catalyst and the metal oxide by direct contact with the oxygen containing gas preferably outside of the reactor as discussed below.

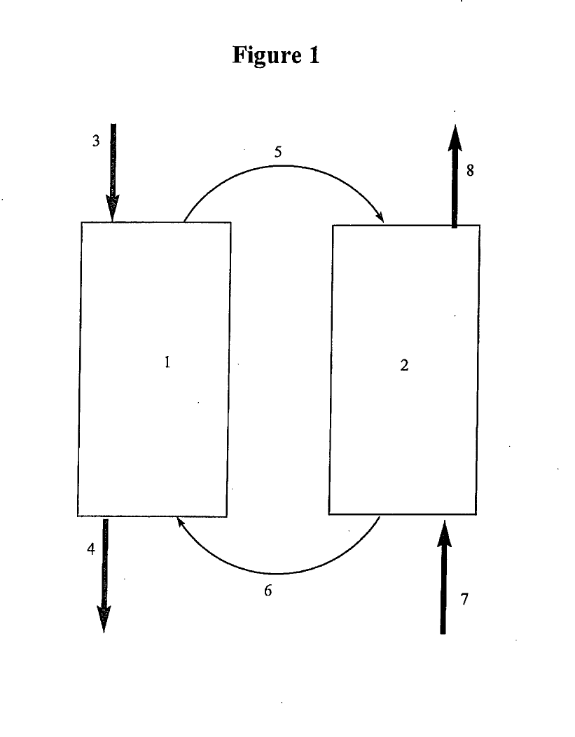

An alternative embodiment is shown in FIG. 1. In FIG. 1 there are two vessels, 1 and 2, in parallel arrangement. In vessel 1 there is a bed, preferably of fluidized bed, or simple moving bed of an oxidative dehydrogenation catalyst and a metal oxide or a metal oxide mixture. A stream of reactants 3, typically paraffin such as ethane, optionally with an inert gas such as nitrogen enters reactor 1. The paraffin undergoes oxidative dehydrogenation and the oxidative dehydrogenation catalyst and the metal oxide mixture gives up oxygen. A stream 4 of alkene such as ethylene leaves the reactor. The bed (or at least the metal oxide component) is moved from reactor 1 to reactor 2 by line 5. An oxygen containing stream 7 such as air enters reactor 2. The oxygen in the oxygen containing stream 7 contacts the depleted oxidative dehydrogenation catalyst and metal oxide or the metal oxide mixture and regenerates them by oxidation. The regenerated oxide or metal oxide mixture and the oxidative dehydrogenation catalyst are then returned to reactor 1 by line 6.

The resulting alkene may be used in any conventional industrial application such as polymerization, the manufacture of glycols or alkylation (e.g. benzene to ethyl benzene).

The process of the present invention is practiced at temperatures lower than the conventional cracking processes reducing energy costs and green house gases. Additionally if the feed is a relatively pure alkane (ethane) and a oxidative dehydrogenation catalyst is used which has a high selectivity (e.g. greater than 95%, preferably greater than 98%) for the corresponding 1-alkene the back end separation costs are significantly reduced over the current cryogenic back end separation cost for thermal cracking. Potentially the resulting stream of alkene and alkane could be used in the dilute ethylene processes as illustrated by U.S. Pat. Nos. 5,981,818 issued Nov. 9, 1999 and 6,111,156 issued Aug. 19, 2000. Again this reduces energy consumption.

The process of the present invention will now be illustrated by the following non limiting examples.

### EXAMPLES

### Example 1

### Preparation of the Active Oxide Catalyst Phase, No Support

2.65 g of ammonium heptamolybdate (tetrahydrate) and 0.575 g of telluric acid were dissolved in 19.5 g of distilled water at 80° C. Ammonium hydroxide (25% aqueous solution) is added to the Mo- and Te-containing solution to obtain a pH of 7.5. Then water is evaporated under stirring at 80° C. The solid precipitate is dried at 90° C. 3.0 g of this precipitate is suspended in water (21.3 g) at 80° C. and 0.9 g of vanadyl sulfate and 1.039 g of niobium oxalate were added. The mixture was stirred for 10 min and then is transferred to the autoclave with a Teflon® (tetrafluoroethylene) lining. Air in the autoclave was replaced with argon, the autoclave was pressurized and heated to 175° C. and the system was kept for 60 hours at this temperature. Then the solid formed in the autoclave was filtered, washed with distilled water and dried at 80° C. The thus obtained active catalyst phase was calcined at 600° C. (2 h) in a flow of argon. The temperature was ramped from room temperature to 600° C. at 1.67° C./min. The powder was pressed then and the required mesh size particles were collected.

### Catalyst Activity

The catalyst was tested in oxidative dehydrogenation of ethane using a gas mixture O₂/C₂H₆ with an O₂ content of 25% (outside the explosive limit). The mixture was fed in the plug-flow reactor with the gas hourly space velocity of 900 h⁻¹ at a pressure of 1 atm.

The catalysts were tested at 420° C., the catalyst loading 0.13-1.3 g; fraction (particle size) 0.25-0.5 mm, a flow type reactor with a stationary catalyst bed was used. The catalyst was heated to 360° C. in the reaction mixture and the catalytic activity was measured at 420° C. The data are presented in the Table 1 (Entry 1)

### Example 2

### Moving Bed Reactor

The active catalyst prepared in Example 1 was placed in a moving bed reactor and was tested in oxidative dehydrogenation of ethane by varying the residence time of the alkane feed to the reactor while keeping the temperature at 420° C.

The results of the experiments are set forth in Table 1 below. The catalyst performances are given for the V—Mo—Nb—Te—O catalyst in oxidative dehydrogenation of ethane at 420° C. in conventional mode (direct oxidation of a feed which is a mixture of 75% ethane and 25% oxygen and in moving bed mode separate flows of pure ethane and air to the moving bed reactor to different zones to re generate the oxidative dehydrogenation catalyst and oxidative dehydrogenate the ethane at a space velocity of 900 hr⁻¹.

TABLE 1

| | Residence | Space-time yield of | |
|---|---|---|---|
| | time | ethylene (Productivity). | Ethylene |
| Example | Seconds | g/h per kg of catalyst | Selectivity % |
| 1 (Comparative) | 4 | 210 | 90-92 |
| 2 | 4 | 980 | 96 |
| | 2 | 1,800 | 97 |
| | 1 | 3,500 | 98 |

These results show the enhancement in ethylene time-space yield at shorter residence times, demonstrating that the catalyst is releasing oxygen to the oxidative dehydrogenation process to increase the space time yield and the selectivity.

What is claimed:

1. A process for the oxidative dehydrogenation of one or more C₂₋₁₀ alkanes to the corresponding C₂₋₁₀ alkene at a selectivity of greater than 95%, comprising contacting said alkane in the absence of a gaseous oxygen with a moving or fluid particulate bed of oxidative dehydrogenation catalyst having an enhanced labile oxygen content in the crystal structure on an inert support optionally with a regenerable metal oxide composition at a temperature from 300° C. to 700° C., a pressure from 0.5 to 100 psi (3.447 to 689.47 kPa) and a residence time of the alkane in said bed from 1 to 60 seconds, wherein the oxidative dehydrogenation catalyst is selected from the group consisting of
i) catalysts of the formula:
VₓMoₐNb_{b}Te_{c}Me_{d}Oₑ,
wherein
Me is a metal selected from the group consisting of Ti, Ta, Sb, Hf, W, Y, Zn, Zr, La, Ce, Pr, Nd, Sm, Sn, Bi, Pb Cr, Mn, Fe, Co, Cu, Ru, Rh, Pd,
Pt, Ag, Cd, Os, Ir, Au, and mixtures thereof; and
x is from 0.1 to 0.9;
a is from 0.001 to 0.5;
b is from 0.001 to 0.5;
c is from 0.001 to 0.5;
d is from 0.001 to 0.5
and e is a number to satisfy the valence state of the mixed oxide catalyst and regenerating at least one of the labile oxygen content in the crystal structure of the oxidative dehydrogenation catalyst and the metal oxide if present.

2. The process according to claim 1 wherein the temperature is from 350° C. to 500° C., the pressures is from 15 to 50 psi (103.4 to 344.73 kPa) and the residence time of the alkane in said bed is from 2 to 20 seconds.

3. The process according to claim 2, wherein the oxidative dehydrogenation catalyst is on a support selected from the group consisting of oxides of titanium, zirconium, aluminum, magnesium, yttrium, lanthanum, silicon and their mixed compositions or a carbon matrix at a loading from 1 to 95 weight % of the supported oxidative dehydrogenation catalyst.

4. The process according to claim 3, wherein the oxidative dehydrogenation catalyst has a selectivity for the corresponding 1-alkene of greater than 95%.

5. The process according to claim 4, wherein the space-time yield of alkene in g/hour per Kg of catalyst is greater than 900 g/hour per kg of oxidative dehydrogenation catalyst.

6. The process according to claim 5, wherein the alkane is selected from the group consisting of C₂₋₄ straight chained alkanes.

7. The process according to claim 6, wherein the regeneration of the catalyst and metal oxide when present takes place at temperatures from 200° C. to 600° C., at pressures less than 10132.5 kPa (100 atm, 14700 psi) and the gaseous feed stream for the regeneration is selected from the group consisting of air, oxygen or a mixture of about 10 to 45 wt % oxygen and from 90 to 55 wt % of an inert gas.

8. The process according to claim 7, wherein the alkane is ethane, and in the catalyst x is from 0.02 to 0.5, a is from 0.1 to 0.45, b is from 0.1 to 0.45, c is from 0.1 to 0.45, and d is from 0.1 to 0.45.

9. The process according to claim 8, wherein there are two or more separate fixed beds in parallel arrangement and one or more beds is regenerated by passing air therethrough while maintaining at least one bed in operation.

10. The process according to claim 8 wherein the bed is a fluidized bed or a simple moving bed and a portion of the bed is removed from the reactor and regenerated by passing air therethrough and the regenerated bed is returned to the reactor.

11. The process according to claim 9, where in the metal oxide is present and is selected from the group consisting of NiO, Ce₂O, Ce₂O₃, Fe₂O₃, TiO₂, Cr₂O₃, V₂O₅, WO₃, and ferrites of the formula MFeO₄ where M is selected from the group consisting of Mf, Mn, Co, Ni, Zn, or Cd, and alumina and mixtures and is present in an amount to provide a weight ratio of oxidative dehydrogenation catalyst to metal oxide from 0.8:1 to 1:0.8.

12. The process according to claim 10, wherein the metal oxide is present and is selected from the group consisting of NiO, Ce₂O₃, Fe₂O₃, TiO₂, Cr₂O₃, V₂O₅, WO₃ and ferrites of the formula MFeO₄ where M is selected from the group consisting of Mf, Mn, Co, Ni, Zn, or Cd, and alumina and mixtures and is present in an amount to provide a weight ratio of oxidative dehydrogenation catalyst to metal oxide from 0.8:1 to 1:0.8.

13. The process according to claim 8, wherein the metal oxide is selected from the group consisting of NiO, Ce₂O₃, Fe₂O₃, TiO₂, Cr₂O₃, V₂O₅, WO₃, and ferrites of the formula MFeO₄ where M is selected from the group consisting of Mf, Mn, Co, Ni, Zn, or Cd, and alumina and mixtures and is present in an amount to provide a weight ratio of oxidative dehydrogenation catalyst to metal oxide from 0.8:1 to 1:0.8.

14. The process according to claim 13, wherein the bed is a segregated bed with the metal oxide separated from the oxidative dehydrogenation catalyst by a screen or an oxygen permeable membrane and at least a portion of the metal oxide is removed from said bed and regenerated by passing an oxygen containing gas stream therethrough and the metal oxide is returned to the bed.
